(19) United States
(12) Patent Application Publication
(10) Pub. No.: US 2005/0036991 A1
(43) Pub. Date: Feb. 17, 2005

(54) Mixtures of caspase inhibitors and complement inhibitors and methods of use thereof

(76) Inventor: William L Fodor, Madison, CT (US)

Correspondence Address: Mark Farber;Alexion Pharmaceuticals Inc, 352 Knotter Drive, Cheshire, CT, 06410, US

(21) Appl. No.: 10/203,676

(22) PCT Filed: Feb. 9, 2001
(86) PCT No.: PCT/US01/04137

Related U.S. Application Data
(60) Provisional application No. 60/181,478, filed on Feb. 10, 2000.

(51) Int. Cl. A61K 45/00; A01N 1/02; C12N 5/08

(52) U.S. Cl. 424/93.7; 435/2; 435/366

(57) ABSTRACT

Combinations of caspase inhibitors and complement inhibitors are described. Methods for preventing and/or treating transplant rejection, and in particular rejection of xenotransplants, involving treating the graft material and/or the transplant recipient with a combination of a caspase inhibitor and a complement inhibitor are also described.

### TECHNICAL FIELD

Mixtures of caspase inhibitors and complement inhibitors and pharmaceutically acceptable compositions containing the mixtures are described. Methods for preventing and/or treating transplant rejection, and in particular rejection of xenotransplants, involving treating the graft material and/or the transplant recipient with a combination of a caspase inhibitor and a complement inhibitor are also described.

### BACKGROUND

One of the major challenges encountered in transplantation methodology is the rejection of transplanted organs and tissues due to the natural humoral and cellular immunologic mechanisms of the host. For example, in the area of fetal neural cell transplantation as a dopaminergic replacement therapy for Parkinson's disease, it is reported in the literature that up to 99% of the transplanted neurons die during graft development. See Nature 362: 414-15, Acta Physiol. Scand. Suppl. 522: 1-7, Neurosci. Lett. 61: 79-84, and Brain Res. 331: 251-59. The types of cell death that have been observed in transplanted fetal grafts include apoptosis (or programmed cell death), necrosis, cellular immune-mediated and complement mediated cytolysis. There are clear benefits to preventing the amount of cell loss seen in neural transplants, such as improving functional effects, reducing inflammation, and the presence of immunological stimuli that could lead to transplant rejection. For practical purposes there is a necessity to reduce the amount of transplantable tissue needed to achieve functional effects in the recipient, e.g. 10-15 fetuses are required to obtain a set of transplantable ventral mesencephalic cells for a single Parkinson's patient.

An important difference between apoptosis and necrosis of neurons is that the former is under active cell control. Research on the nematode Caenorhabditis elegans has led to the understanding that apoptosis is evolutionary and genetically conserved (Cell 75: 641-652), as well as the identification of pro- and antiapoptotic genes for which there are mammalian homologues. For example, the ced-3 gene in C. elegans encodes a member of the ICE cysteine protease family homologous to caspase-3-like caspases which is vital for the execution of all programmed cell deaths in mammals. The caspases, a family of 12 cysteine proteases, are synthesized as inactive proenzymes in the cytoplasm and are activated by cleavage at internally specified conserved aspartate residues. Once activated, the caspases initiate a cascade of ultracellular proteolytic cleavage events leading to activation of downstream caspases with cellular substrates. For example, activation of the inactive pro-caspase-3 to the active caspase-3 occurs by the release of cytochrome c from the mitochondria that are under the influence of other cellular apoptotic mechanisms. Caspase-3 cleaves other caspases in the death cascade.

Pharmacological inhibition of caspases as a means to decrease cell death in neural transplants is known, see for example, the review article entitled “Apoptosis in Neuronal Development and Transplantation: Role of Caspases and Trophic Factors”, Exp. Neurol. 156: 1-15 (1999). Treatment of dissociated cell suspension or dissected tissue pieces with caspase inhibitors prior to transplantation into the host brain is one strategy set forth in the review article (Ibid., at page 7) Included in the review, is a summary of in vitro and in vivo studies that been carried out with the following caspase inhibitors and aimed at neuroprotection by decreasing apoptosis: z-VAD-DCB (an irreversible ICE/caspase-1 inhibitor), z-DEVD-fmk (a rather specific inhibitor of caspase-3), viral caspase inhibitor gene p35 and broad spectrum caspase inhibitor benzyloxycarbonyl-Val-Ala-Asp-fluoromethylketone (z-VAD.fmk) (inhibiting caspase-3 or caspase-3-like proteases), acetyl-DEVD-CHO (specific caspase-3 inhibitor), Bocaspartyl(OMe)-fluoromethylketone (BAF) (inhibitor of caspase-1 and caspase-3), and caspase-1-specific inhibitors, e.g., Ac-Try-Val-Ala-Asp-chloromethylketone (Y-VAD.CMK), Ac-Try-Val-Ala-Asp-aldehyde, and crmA (a cytokine response modifier gene and a viral caspase inhibitor). The review article suggests possible combinations of caspase inhibitors with trophic factors in neural transplants to block cell death.

The phenomenon of hyperacute rejection (also referred to as “HAR”) is typified by an antibody-primed, complement-mediated graft rejection that is usually rapid and irreversible. HAR is encountered in xenotransplanted organs (donor organ is from a different species), and to a lesser degree in allogeneic transplants. HAR is initiated by the deposition of natural or induced antibodies on donor endothelium followed by the activation of the recipient complement system which rapidly destroys the graft. More specifically, studies have suggested that activated early complement components such as C3a and C3b and late complement components, such as C5a and C5b-9 membrane attack complex (MAC), as well as natural antibody deposition may contribute directly to xenograft rejection. Before describing complement-targeted strategies for decreasing HAR, the complement system is briefly summarized.

The complement system is a complex interaction of at least 25 plasma proteins and membrane cofactors which act in a multi-step, multi-protein cascade sequence in conjunction with other immunological systems of the body to provide immunity from intrusion of foreign cells and viruses. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16^{th} Edition.

There are two complement pathways, the classical pathway and the alternative pathway. The classical pathway which is usually initiated by antigen-antibody (Ag-Ab) complexes, wherein certain of the antibodies are complement fixing or capable of binding to complement to activate the pathway. The alternative complement pathway is usually antibody independent and can be initiated by certain molecules on pathogen surfaces. While both pathways proceed along distinct cascade events initially, both classical and alternative complement activation merge at the single most important step of cleavage of C3 into C3a and C3b, by the respective C3 convertases produced by each pathway. There is a single final pathway known as the terminal pathway, or the membrane attack complex (also referred to as “MAC”). The formation of MAC begins with formation of the cleavage product C5b derived from the action of C5 convertase on C5. The C5 convertase is formed from a C3 convertase. Towards the end of an intricate series of numerous complexation events, component C9 binds to a complex designated as C5b,6,7,8 to form C5b-9 or MAC, and results in substantial cell lysis and/or other effects such as deleterious cell activation, e.g., as described in Transplantation 60 (11): 1284-92, at 1285 (1995). Additional C9 binds with C5b-9 to cause increased rate of lysis.

Studies reported in the literature have demonstrated that HAR does not occur in settings where the MAC cannot be formed, either by inhibition of complement activation prior to MAC formation (e.g., by removal of xenoreactive natural antibodies, depletion of complement with cobra venom factor, or inhibition of complement using soluble CR1) or by using functionally blocking monoclonal antibodies directed against, e.g., the human MAC components C5 and C8. Transplantation 60 (11) at page 1285. Accordingly, anti-C5 and anti-C8 mAbs are known.

Also, cell-surface-bound complement regulatory (inhibitory) proteins, such as CD59, are described in the family of related patents beginning with parent U.S. Pat. No. 5,135,916 (assigned to Oklahoma Medical Research Foundation), and inhibit C5b-9 complex assembly. Also included in this family of patents are antibodies or active fragments thereof that mimic the inhibitory action of the inhibitory protein, as well as monoclonal antibodies that specifically bind to a component of the C5b-9 complex, e.g., anti-C7 and anti-C9 mAbs. A family of cell-surface proteins that regulate or inhibit the crucial C3b cleavage component are membrane cofactor protein (MCP or CD46), decay accelerating factor (DAF or CD55), complement receptor 1 (CR1 or CD55), factor H and C4b-binding protein and are disclosed, e.g., in U.S. Pat. No. 5,705,732.

Another class of inhibitor proteins are the chimeric complement inhibitor proteins that contain functional domains from two complement inhibitor proteins, such as C3 inhibitor proteins and C5b-9 inhibitor proteins. These are described, e.g., in U.S. Pat. Nos. 5,624,837, 5,627,264, and 5,847,082 (all assigned to Alexion Pharmaceuticals, Inc.) In spite of the current knowledge pertaining to increasing cell survival of xenografts and allografts by either inhibition of the recipient complement system or by controlling apoptosis or programmed cell death, the benefit of using combinations of a caspase inhibitor and a complement is hereto for unrecognized in the art.

### SUMMARY

It has now surprisingly been found that a combination of at least one caspase inhibitor and at least one complement inhibitor can be used in the treatment and/or prevention of transplant rejection. The combination can be used to treat cellular material to be transplanted before or during transplantation. In an alternative embodiment, the at least one complement inhibitor is administered systematically to a transplant recipient before, during and/or after transplantation of cellular material that has been pre-treated with at least one caspase inhibitor or treated with a combination of at least one caspase inhibitor and at least one complement inhibitor.

In one embodiment, the transplant cells, tissues, or organs, are treated with a solution containing at least one caspase inhibitor in an amount of between about 1 to about 10 μM final and are then prepared as a cell suspension containing complement inhibitor in an amount from about 50 to about 500 μg/ml of cell suspension.

### BRIEF DESCRIPTION OF THE DRAWINGS

FIG. 1A is a photomicrograph (40×) of xenografted fetal pig cells into rat striata, which have been pre-treated with the caspase inhibitor bocaspartyl(o-methyl)-flouromethylketone before transplantation. Immumohistochemical staining was performed with a pig specific neurofilament 70 kd antibody (NF70) following transplantation and tissue harvest.

FIG. 1B is a photomicrograph (40×) of xenografted fetal pig cells into rat striata, which have been pre-treated with an anti-C-5 antibody before transplantation. Immumohistochemical staining was performed with a pig specific neurofilament 70 kd antibody (NF70) following transplantation and tissue harvest.

FIG. 1C is a photomicrograph (40×) of xenografted fetal pig cells into rat striata, which have been pre-treated with a mixture of the caspase inhibitor bocaspartyl(o-methyl)-flouromethylketone and an anti-C-5 antibody before transplantation. Immumohistochemical staining was performed with a pig specific neurofilament 70 kd antibody (NF70) following transplantation and tissue harvest.

FIG. 1D is a photomicrograph (40×) of a control group of xenografted fetal pig cells into rat striata. Immumohistochemical staining was performed with a pig specific neurofilament 70 kd antibody (NF70).

FIG. 2 is a graph showing the average striatal graft volume (in mm³) determined by NF70 staining.

FIG. 3 is a graph showing the total number of TH positive cells in striatal graft sites per group.

### DESCRIPTION OF THE PREFERRED EMBODIMENTS

It has been found that caspase inhibitors and complement inhibitors can advantageously be used in combination to inhibit transplant rejection. The use of a combination of caspase inhibitors and complement inhibitors has been found to be superior to treatment with either caspase inhibitors or complement inhibitors alone.

Suitable caspase inhibitors include any compound or composition having inhibitory activity to one or more caspase enzymes reactive with the type of cell, tissue, or organ to be transplanted. Such caspase inhibitors include, but are not limited to, z-VAD-DCB (an irreversible ICE/caspase-1 inhibitor), z-DEVD-fmk (a rather specific inhibitor of caspase-3), viral caspase inhibitor gene p35 and broad spectrum caspase inhibitor benzyloxycarbonyl-Val-Ala-Asp-fluoromethylketone (z-VAD.fmk) (inhibiting caspase-3 or caspase-3-like proteases), acetyl-DEVD-CHO (specific caspase-3 inhibitor), Bocaspartyl(OMe)-fluoromethylketone (BAF) (inhibitor of caspase-1 and caspase-3), and caspase-1-specific inhibitors, e.g., Ac-Try-Val-AIa-Asp-chloromethylketone (Y-VAD.CMK), Ac-Try-Val-Ala-Asp-aldehyde, crmA (a cytokine response modifier gene and a viral caspase inhibitor), Ac-WAD-cmk (an inhibitor of caspase 1), CPP (an inhibitor of caspases 1 and 3) and z-DEVD-fmk (an inhibitor of caspase 3). Other known caspase inhibitors can be used such as those disclosed in U.S. Pat. Nos. 6,153,591 and “Apoptosis in Neuronal Development and Transplantation: Role of Caspases and Trophic Factors”, Exp. Neurol. 156: 1-15 (1999), the contents of which are incorporated herein by reference. It should be understood that combinations of caspase inhibitors can be employed in the compositions and methods described herein. Preferably, the caspase inhibitor is not specific to one caspase. Particularly useful caspase inhibitors are bocaspartyl(o-methyl)-flouromethylketone (BAF) and Ac-YVAD-cmk.

Any compounds which bind to or otherwise block the generation and/or activity of any of the human complement components, such as, for example, antibodies specific to a human complement can be used as the complement inhibitor in the compositions and methods described herein. Some useful complement inhibitor compounds include 1) antibodies directed against complement components C-1, C-2, C-3, C-4, C-5, C-6, C-7, C-8, C-9, Factor D, Factor B, Factor P, MBL, MASP-1, AND MASP-2 and 2) naturally occurring or soluble forms of complement inhibitory compounds such as CR1, LEX-CR1, MCP, DAF, CD59, Factor H, cobra venom factor, FUT-175, y bind protein, complestatin, and K76 COOH. Suitable compounds for use herein are antibodies that reduce, directly or indirectly, the conversion of complement component C5 into complement components C5a and C5b. One class of useful antibodies are those having at least one antibody-antigen binding site and exhibiting specific binding to human complement component C5, wherein the specific binding is targeted to the alpha chain of human complement component C5. Such an antibody 1) inhibits complement activation in a human body fluid; 2) inhibits the binding of purified human complement component C5 to either human complement component C3 or human complement component C4; and 3) does not specifically bind to the human complement activation product for C5a. Particularly useful complement inhibitors are compounds which reduce the generation of C5a and/or C5b-9 by greater than about 30%. A particularly useful anti-C5 antibody is h5G1.1-scFv. Methods for the preparation of h5G1.1-scFv are described in U.S. patent application Ser. No. 08/487,283 filed Jun. 7, 1995 now U.S. Pat. No. ______ and “Inhibition of Complement Activity by Humanized Anti-C5 Antibody and Single Chain Fv”, Thomas et al., Molecular Immunology, Vol. 33, No. 17/18, pages 1389-1401, 1996, the disclosures of which are incorporated herein in their entirety by this reference.

Suitable complement inhibitors include antibodies against C1, C2, C3, C4, C5, C6, C7, C8, and C9, such as those disclosed in U.S. Pat. Nos. 5,635,178; 5,843,884; 5,847,082; 5,853,722; and in Rollins et al.; Monoclonal Antibodies Directed Against Human C5 and C8 Block Complement-Mediated Damage of Xenogeneic Cells and Organs; Transplantation, Vol.60, 1284-1292, 1995; the contents of all of which are incorporated herein by reference. As used herein, the term “antibodies” refers to 1) immunoglobulins produced in vivo; 2) those produced in vitro by a hybridoma; 3) antigen binding fragments (e.g., Fab' preparations) of such immunoglobulins; and 4) recombinantly expressed antigen binding proteins (including chimeric immunoglobulins, bispecific immunoglobulins, heteroconjugate immunoglobulins, “humanized” immunoglobulins, single chain antibodies, antigen binding fragments thereof, and other recombinant proteins containing antigen binding domains derived from immunoglobulins). Such antibodies can include, but are not limited to, polyclonal, monoclonal, humanized, bispecific, and heteroconjugate antibodies and can be prepared by applying methods known in the art. See for example; Reichmann, et al., Nature 332, pp. 323, 1988. Winter and Milstein,1991; Clackson, et al., Nature 352, pp. 624. 1991; Morrison, Annu Rev Immunol 10, pp. 239; 1992; Haber, Immunol Rev 130, pp. 189; 1992; and Rodrigues, et al., J Immunol 151, pp. 6954; 1993.

Suitable polyclonal antibodies can be prepared by methods known to one skilled in the art and the immunization protocol may be selected without undue experimentation. Suitable methods for raising the polyclonal antibodies to C1, C2, C3, C4, C5, C6, C7, C8, and C9 in a mammal include injecting the mammal with an immunizing agent and optionally in the presence or absence of an adjuvant. The regimen includes multiple subcutaneous or interperitoneal injections with the immunizing agent, such as C5 or fragments thereof. It may be useful to conjugate the immunizing agent to a carrier known to be immunogenic in the mammal being immunized.

Suitable monoclonal antibodies may be prepared by using methods to generate hybridomas such as those described in Kohler et al, Nature, 256:495 (1975). Briefly, a mouse, hamster, or other suitable host is immunized with an immunizing agent to elicit lymphocytes that produce or are capable of producing antibodies that will bind to the immunizing agent. The lymphocytes may also be activated to produce antibodies immunized in vitro. The lymphocytes are then fused to myeloma cells in vitro to immortalize the antibody-producing cells.

Techniques for the following are all known in the art: 1) immunization of animals (in one embodiment with C5 fragments thereof), isolation of antibody producing cells, 2) fusion of such cells with immortal cells (e.g., myeloma cells) to generate hybridomas secreting monoclonal antibodies, 3) screening of hybridoma supernatants for reactivity and/or lack of reactivity of secreted monoclonal antibodies with particular antigens, 4) the preparation of quantities of such antibodies in hybridoma supernatants or as-cites fluids, and 5) the purification and storage of such monoclonal antibodies. See for example, Coligan, et al., eds. Current Protocols In Immunology, John Wiley & Sons, New York, 1992; Harlow and Lane, Antibodies, A Laboratory Manual, Cold Spring Harbor Laboratory, New York, 1988; Liddell and Cryer, A Practical Guide To Monoclonal Antibodies, John Wiley & Sons, Chichester, West Sussex, England, 1991; the contents of all of which are incorporated herein by reference.

Humanized anti C1, C2, C3, C4, C5, C6, C7, C8 and C9 antibodies can also be used as the complement inhibitor. Humanized forms of non-human (e.g., murine) antibodies are chimeric immunoglobulins, immunoglobulin chains or fragments thereof (such as Fv, scFv, Fab, Fab′, (Fab′)² or other antigen-binding subsequences of antibodies) which contain minimal sequence derived from non-human immunoglobulin. Humanized antibodies include human immunoglobulins (recipient antibody) in which residues from complementary determining regions (CDRs) of the recipient are replaced by residues from CDRs of a non-human species (donor antibody) such as mouse, rat or rabbit having the desired specificity, affinity and binding capacity. In some instances, specific Fv framework residues of the human immunoglobulin are replaced by corresponding non-human residues. Humanized antibodies may also comprise residues which are found neither in the recipient antibody nor in the imported CDR or framework sequences.

Methods for humanizing non-human antibodies are well known in the art. Generally, a humanized antibody contains one or more amino acid residues that are introduced from a non-human antibody source. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al. J. Mol. Biol., 222:581 (1991)]. The techniques of Cole et al. and Boerner et al. are also available for the preparation of human monoclonal antibodies [(Cole et al., Monoclonal Antibodies and Cancer Therapy, Alan R. Liss p. 77(1985) and Boerner et al., J. Immunol. 147(1):86-95(1991)].

Similarly, human antibodies can be made by introducing human immunoglobulin loci into transgenic animals, (e.g., mice) in which the endogenous immunoglobulin genes have been partially or completely inactivated. Upon challenge with antigens, only human antibodies are produced in a manner similar to that seen in humans in all respects, including gene rearrangement, assembly, and antibody repertoire. See for example, in U.S. Pat. Nos. 5,545,807; 5,545,806; 5,569,825; 5,625,126; 5,633,425; 5,661,016, and in the following scientific publications; Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826(1996); Lonberg and Huszar, Intern. Rev. Immunol. 13 65-93(1995).

Polyspecific antibodies monoclonal, preferably human or humanized, antibodies that have binding specificities for at least two different antigens are also provided. One of the binding specificities, for example, may be specific to C5, while the other may be for any other antigen, cell-surface protein, receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains and/or the two light chains have different specificities (See Milstein and Cuello, Nature, 305:537-539 (1983)). The purification of the correct molecule is usually accomplished by affinity chromatography steps. Similar procedures are disclosed in Traunecker et al., EMBO J. 10:3655-3659 (1991).

Heteroconjugate antibodies, composed of two covalently joined antibodies, are also provided. Such antibodies have, for example, been proposed to link immune system cells to unwanted target cells to enable their rapid elimination (See, U.S. Pat. No. 4,676,980), and to treat HIV infection (See, WO 91/00360; WO 92/200373; and EP 03089). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Other suitable complement inhibitors include molecules having a C5b-9 inhibitory domain and a C3 inhibitory domain.

Suitable domains which exhibit C5b-9 inhibitory activity (as used herein, the phrase “C5b-9 inhibitory activity” describes the effects of C5b-9 inhibitor molecules on the complement system and thus includes activities that lead to inhibition of the cell activating and/or lytic function of the membrane attack complex, hereinafter referred to as MAC) can include the entire amino acid sequence for a naturally occurring C5b-9 inhibitor protein or a portion thereof. For example, the C5b-9 sequence can be the mature CD59. Alternatively, the C5b-9 sequence can be a portion of a naturally occurring C5b-9 inhibitor protein, such as CD59. Active portions suitable for use herein can be identified using a variety of assays for C5b-9 inhibitory activity known in the art. See for example Rollins, et al., J. Immunol. 144:3478, 1990; Rollins, et al., J. Immunol. 146:2345,1991; Zhao, et al., J. Biol. Chem. 266: 13418, 1991; and Rother, et al., J. Virol. 68:730, 1994. In general, the portion used should have at least about 25% and preferably at least about 50% of the activity of the parent molecule.

Suitable C3 inhibitory domains include the entire amino acid sequence for a naturally occurring C3 inhibitor or a portion thereof, such as one or more SCRs of the C3 inhibitory domain. For example, the C3 sequence can be the mature DAF molecule. Alternatively, the C3 inhibitory domain can be a portion of a naturally occurring C3 inhibitor protein. Following the procedures used to identify functional domains of DAF (Adams, et al., 1991. J. Immunol. 147:3005-3011), functional domains of other C3 inhibitors can be identified and used herein. In general, the portion used should have at least about 25% and preferably at least about 50% of the activity of the parent C3 inhibitory molecule. Particularly useful portions of mature C3 inhibitor proteins include one or more of the mature molecule's SCRs. These SCRs are normally approximately 60 amino acids in length and have four conserved cysteine residues which form disulfide bonds, as well as conserved tryptophan, glycine, and phenylalanine/tyrosine residues. One such the C3 inhibitory domain includes SCRs 2 through 4 of DAF.

Molecules having C5b-9 inhibitory activity and/or C3 inhibitory activity are disclosed in for example U.S. Pat. Nos. 5,135,916; 5,179,198; 5,521,296; 5,573,940; 5,627,264; 5,624,9837; 5,573,940; 5,705,732; 5,847,082; and EP394035 the contents of all of which are incorporated herein by reference.

A combination of caspase inhibitors and complement inhibitors can be used for the prevention or treatment of transplant rejection, and preferably xenotransplant rejection. In one embodiment, cellular material to be transplanted (e.g., cells, tissue or organ) is contacted with a solution containing at least one caspase inhibitor and then contacted with a solution containing at least one complement inhibitor. Material so treated can then be transplanted into a recipient.

In contacting the material to be transplanted with caspase inhibitor, a solution containing at least one caspase inhibitor in an amount from about 0.1 μM to about 100 μM, preferably from about 1 μM to about 10 μM final can be used. Preferably, the material to be transplanted is incubated in a solution containing at least one caspase inhibitor for a period of time ranging from about 1 to about 60 minutes, preferably from about 10 to about 30 minutes at a temperature in the range of from about 4 to about 40° C., preferably from about 30 to 40° C. (during trypsinization), and preferably about 4 to 10° C. (after trypsinization). The solution of caspase inhibitor can be prepared using any cell culture medium. A particularly useful solution contains calcium- and magnesium-free Hanks' Balanced Salt Solution (HBSS) (commercially available from Sigma Chemical Co.). Upon contact with the solution of caspase inhibitor, the caspase inhibitor will be internalized into the cells, thereby producing an artificially increased concentration of caspase inhibitor within the cells of the material to be transplanted. Once inside the cells, the caspase inhibitor will find and inhibit the activity of one or more of the caspases.

After contact with the solution containing the caspase inhibitor, the cells or tissue to be transplanted can be washed to remove any excess solution of the caspase inhibitor. Any cell culture medium can be used to wash the material to be transplanted. A particularly useful solution contains HBSS, DNAse (such as Pulmozyme, recombinant human DNAse commercially available from Genentech) and glucose. The material to be transplanted can be washed from one to ten times, preferably from 2 to 5 times to remove excess caspase inhibitor. If desired, one or more of the wash solutions can contain a solution of caspase inhibitor in DMSO.

Where the material to be transplanted consists of individual or small aggregates of cells, the washed cells are then used to prepare a cell suspension containing at least one complement inhibitor. The cell suspension advantageously contains from about 10,000 cells/ml to about 300,000 cells/ml, preferably from about 75,000 cells/ml to about 150,000 cells/ ml. The concentration of cells used in the cell suspension will depend on a number of factors including but not limited to the type of cells being transplanted. The amount of complement inhibitor employed in the cell suspension should be at least an amount sufficient to block complement activity in an in vitro cell lysis assay. One suitable assay is the cell lysis assay described in U.S. Pat. No. 6,074,642, the disclosure of which is incorporated herein by reference. The amount of complement inhibitor present in the suspension will depend on a number of factors including but not limited to the specific complement inhibitor chosen. Typically, however, the complement inhibitor will be present in the cell suspension in an amount from about 1 μg/ml to about 1,000 μg/ml of cell suspension, preferably, an amount from about 20 μg/ml to about 500 μg/ml of cell suspension, most preferably an amount from about 50 μg/ml to about 300 μg/ml of cell suspension. Any cell culture medium can be used to prepare the cell suspension. A particularly useful suspension contains HBSS, DNAse and glucose.

Where the material to be transplanted is composed of larger aggregates of cells, such as tissue or organs, the material to be transplanted can optionally be contacted with a solution containing at least one complement inhibitor. The amount of complement inhibitor employed in the solution should be at least an amount sufficient to block complement activity in an in vitro cell lysis assay, as described above. The exact amount of complement inhibitor present in the solution will depend on a number of factors including but not limited to the specific complement inhibitor chosen. Typically, however, the complement inhibitor will be present in the solution in an amount from about 1 μg/ml to about 1,000 μg/ml of solution, preferably, an amount from about 20 μg/ml to about 500 μg/ml of solution, most preferably an amount from about 50 μg/ml to about 300 μg/ml of solution. Any cell culture medium can be used to prepare the solution. A particularly useful solution contains HBSS, DNAse and glucose. The tissue or organ can be dipped in, basted with or submerged in the solution containing at least one complement inhibitor.

In another embodiment, the recipient of the transplant is treated with at least on complement inhibitor prior to receiving the transplant. In this embodiment the complement inhibitor is administered systemically to the recipient. The complement inhibitor can be administered by methods well known in the art, such as by bolus injection, intravenous delivery, continuous infusion, sustained release from implants, etc. The complement inhibitor may also be entrapped in microcapsules (such as hydroxymethylcellulose or gelatin-microcapsules); liposomes; and other sustained-release matrices such as polyesters, hydrogels(for example, polyhydroxyethylmethacrylate or polyvinylalcohol) or injectable microspheres of biodegradeable materials, such as polymers and copolymers of glycolide, lactide, and/or ethylene glycol. The dosage of complement inhibitor employed will depend on a number of factors including but not limited to the specific complement inhibitor(s) chosen and the type of material being implanted. For example, antibodies prepared as Fab′ or F(ab′)2 fragments are of considerably smaller mass than the equivalent intact immunoglobulins, and thus require lower dosages to reach the same molar levels in the patient's blood. Antibodies with different affinities will also differ in their regarded dosages. The complement inhibitor can systemically administered alone or in combination with known immunosuppressive agents. Suitable immunosuppressive agents include but are not limited to cyclosporin A, FK506, rapamycin and corticosteroids.

Formulations suitable for injection are found in Remington's Pharmaceutical Sciences, Mack Publishing Company, Philadelphia, Pa., 17th ed. (1985). Such formulations must be sterile and non-pyrogenic, and generally will include purified therapeutic complement inhibitor agents in conjunction with a pharmaceutically effective carrier, such as saline, buffered (e.g., phosphate buffered) saline, Hank's solution, Ringer's solution, dextrose/saline, glucose solutions, and the like. The formulations may contain pharmaceutically acceptable auxiliary substances as required, such as, tonicity adjusting agents, wetting agents, bactericidal agents, preservatives, stabilizers, and the like.

The dose will also vary depending on the manner of administration, the particular symptoms of the patient being treated, the overall health, condition, size, and age of the patient, and the judgment of the prescribing physician. Dosage levels of the mixture for human subjects will normally range between about 1 mg per kg and about 100 mg per kg per patient per treatment, preferably between about 5 mg per kg and about 25 mg per kg per patient per treatment.

Subject to the judgment of the physician, a typical therapeutic treatment includes a series of doses, which are usually administered concurrent with the monitoring of clinical endpoints. These may include xenotransplant biopsies or measures of organ function (e.g. for xenotransplanted kidneys, BUN creatines and, proteinuria levels, etc.), with treatment dosage levels adjusted as needed to achieve the desired clinical outcome. For Parkinson's disease, dosage can be based on the patient's CAPIT (Core Assessment Program For Intracerebral Transplantation) evaluation which includes the UPDRS scale of movement disorder. (See, Schumacher, et al., “Transplantation of Embryonic Porcine Mesencephalic Tissue in Patients with PD”, Neurology, 54, pages 1042-50, March 2000.)

The formulations can be distributed in sterile form as articles of manufacture comprising packaging material and the caspase inhibitor/complement inhibitor combination. The packaging material will include a label which indicates that the formulation is for use in the prevention or treatment of transplant rejection, and preferably porcine xenotransplant rejection. Thus, for example, a kit can be provided which contains a solution containing at least one caspase inhibitor and a solution containing at least one complement inhibitor and instructions for contacting cellular material to be transplanted with the two solutions sequentially.

In order that those skilled in the art may be better able to practice the compositions and methods described herein, the following examples are given as an illustration of the treatment of cells and/or tissues prior to transplantation with a caspase inhibitor and a complement inhibitor, as well as, of the superior characteristics of those cells and/or tissues treated with a combination of a caspase inhibitor and a complement inhibitor. It is to be understood that the invention is not limited to the specific details embodied in the examples and further that that commercially available reagents and/or instrumentation referred to in the examples were used according to the manufacturer's instructions unless otherwise indicated.

### EXAMPLE 1

### Fetal Porcine Ventral Mesencephalon Cell Transplantation into Rats with Striatal Lesions

Preparation of Fetal Porcine Ventral Mesencephalon Cells:

Porcine ventral mesencephalon (VM) grafts from embryos were prepared as described earlier (Isacson et al, 1996) with minor modifications. Fetuses were obtained at postinsemination day 28 and the VM was dissected from the surrounding tissue and placed in Dulbecco's phosphate buffered saline (PBS). The suspension of VM fragments was split into three fractions. One fraction was incubated in calcium- and magnesium-free Hanks' Balanced Salt Solution (HBSS) with 0.05% trypsin, 0.53 mM ethylene diamine tetra acetic acid (EDTA) (commercially available from Sigma Chemical Co.) at 37° C. for 10 minutes. The remaining two fractions were treated as described above, however the caspase inhibitors Bocaspartyl(OMe)-fluoromethylketone (BAF) or Ac-Try-Val-Ala-Asp-chloromethylketone (Ac-YVAD.cmk) was added along with the HBSS EDTA trypsin solution. The concentration of each caspase inhibitor was 10 μM. Following trypsinization, the VM samples were washed four times with HBSS with 50 mg/ml DNAse (Pulmozyme, recombinant human DNAse commercially available from Genentech) and glucose. Samples treated with BAF were washed as described above, however the HBSS with DNAse also contained BAF at 10 μM in 0.25% dimethyl sulfoxone (DMSO). Samples treated with Ac-YVAD.cmk were washed as described above, however the HBSS with DNAse also contained Ac-YVAD.cmk at 10 μM in 0.1% DMSO. VM samples were passed through progressively smaller diameter fire-polished glass needles until single cell suspensions were obtained. The VM cells were counted and assessed for viability by fluorescence microscopy using acridine orange-ethidium bromide (Bjorklund, Isacson and Brundin, 1986). VM cells were suspended at 100,000 cells/ml in HBSS, DNAse, Glucose wash solution. The VM cells treated with BAF were suspended in HBSS, DNAse, Glucose wash solution that also contained BAF at 10 μM. The VM cells treated with Ac-YVAD.cmk were suspended in HBSS, DNAse, Glucose wash solution that also contained Ac-YVAD.cmk at 10 μM. The cell suspensions for the relevant experimental groups also contained mouse anti-C5 antibody, 18 A10 (See, Vakeva, et al. “Myocardial Infarction and Apoptosis after Myocardial lschemia and Reperfusion: Role of the Terminal Complement Components and Inhibition by Anti-C5 Therapy”, Circulation, 1998, Jun. 9, 1997(22): pages 2259-67) in an amount of 200 μg/ml of cell suspension.

Transplantation of Porcine VM Cells into Rats:

Adult female Sprague-Dawley rats were subjected to a standard procedure to create unilateral dopamine (DA) depleting lesions in two striatal sites in the medial forebrain bundle (Isacson et al, 1996). After recovery from the procedure the lesions were verified by behavioral testing. One day prior to VM transplantation the rats were treated with 30 mg/kg cyclosporine A (CSA). The rats were anesthetized and then using a 10 ml Hamilton syringe, 1 ml of VM cell suspension was injected at each of the two striatal lesions at a rate of 0.5 ml/minute followed by a 2 minute pause prior to withdrawal of the needle. The transplantation sites were positioned at coordinates relative to bregma: AP=1.0 mm, L=+3.0 mm, V=−5.0 mm and −4.5 mm (ventral to dural), IB=0. All of the rats received CSA at 15 mg/kg for five days post-transplantation.

The experimental groups were as follows:

BAF/C5 Experiment

| Group | Treatment | Animals/Group |
|---|---|---|
| 1 | HBSS/Glucose/DNAse | 10 |
| 2 | BAF | 10 |
| 3 | C5 Antibody | 10 |
| 4 | BAF and C5 Antibody | 10 |

YVAD/C5 Experiment

| Group | Treatment | Animals/Group |
|---|---|---|
| 1 | HBSS/Glucose/DNAse | 10 |
| 2 | YVAD | 10 |
| 3 | C5 Antibody | 10 |
| 4 | YVAD and C5 Antibody | 10 |

Striatal Graft Morphology and Cell Survival:

Assessment of graft survival was performed by standard methods (Isacson et al, 1996). The rats were sacrificed five weeks after surgery by treatment with sodium pentabarbital and were then perfused through the left ventrical with 250 ml of cold heparinized 0.9% saline (1000 units heparin/L) followed by 250 ml of cold 4% paraformaldehyde in PBS (pH 7.4). The brains were harvested, washed for 8 hours in of cold 4% paraformaldehyde in PBS (pH 7.4) and then equilibrated in 30% sucrose in PBS (pH 7.4). A series of frozen 40 mm coronal sections were obtained and stored in PBS. Neuronal survival and graft morphology was assessed by immunostaining by the avidin biotin conjugated peroxidase method (commercially available from Vector Labs, Burlingham, Calif.) for tyrosine hydroxylase (TH). Donor-derived VM cells were visualized by immunostaining using an antibody for pig neurofilament 70 Kd protein (NF70). Briefly, tissue sections were fixed in 50% methanol and 0.3% hydrogen peroxide in PBS for 20 minutes and then rinsed three times in PBS. The fixed sections were then incubated in a 10% normal goat serum (NGS) blocking solution to limit nonspecific antibody binding. Sections were incubated overnight with TH antibodies (commercially available from Pel Freeze, Rogers, Ak.) at a 1:250 dilution or NF70 antibodies (commercially available from BIODESIGN, Kennebunkport, Me.) in a 1:1000 dilution in PBS containing 1% NGS, 1% bovine serum albumin, and 0.1% triton-X 100. The sections were then washed in PBS and then incubated for 90 minutes with the following secondary antibodies. To detect TH, biotinylated goat ant-rabbit antibodies diluted 1:200 in 2% NBS in PBS was added and to detect NF70 biotinylated goat anti-mouse antibodies diluted 1:1000 in 2% NBS in PBS was added. The sections were washed once with PBS and twice in 0.05 mM tris-buffered saline. The secondary antibodies were visualized using a standard avidin-conjugated staining method (Vectastain ABC Kit commercially available from Vecter Labs).

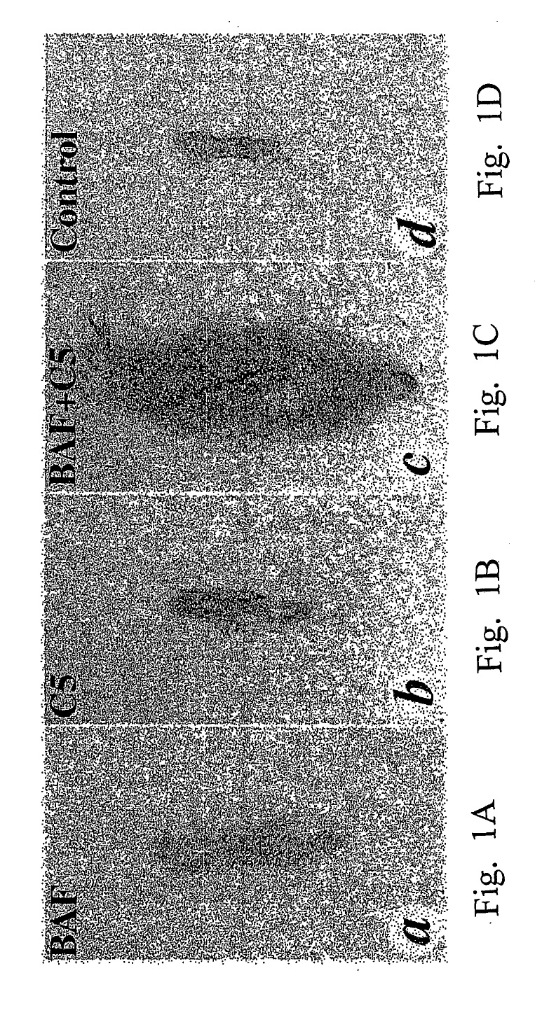

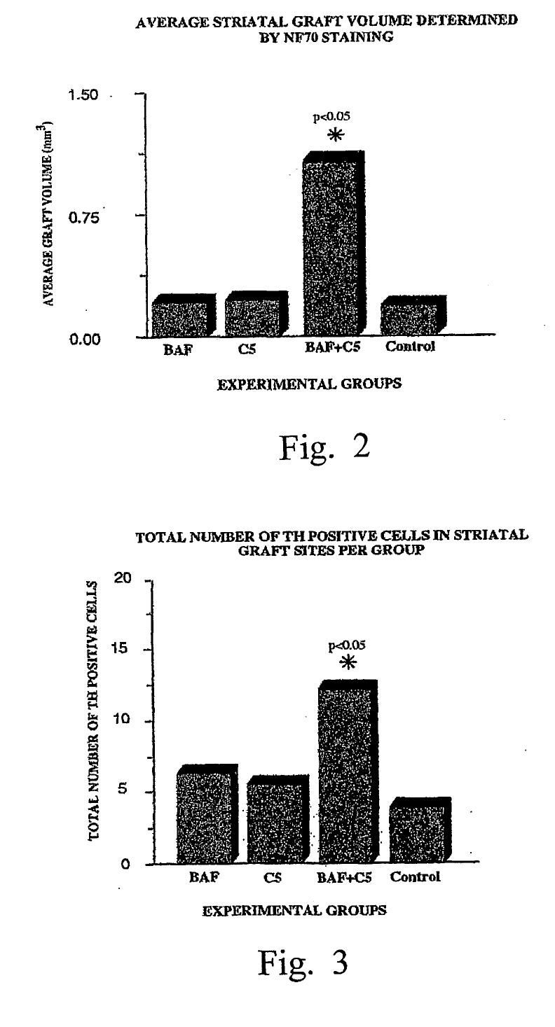

Assessment of sections from the 40 animals in the BAF/C5 experiment revealed that all of the transplant recipients had surviving porcine fetal VM cells, although there was variability in the placement of the grafts. As seen in FIG. 3, the most striking difference in graft volume was observed between the BAF+C5 and Control groups, the former showing larger grafts. The difference was significant according to both the Tukey-Kramer and ANOVA (p<0.025) tests. The graft volume was measured using the NF70-stained sections. Under 10 fold magnification graft images were digitized using Adobe Photoshop and then graft area was determined using NIH Image software. The graft area of a given section was an average of five determinations. The total brain graft area was calculated by multiplying the average section value by the section thickness (40 mm) and then adding all of the section values together. The most striking increase in graft size measured by NF70 staining compared to controls (HBSS/Glucose/DNAse) was observed with BAF/C5 cohort (see FIG. 1C and FIG. 2). The results show that treatment of VM cells with BAF prior to implantation, combined with C5 antibody and postoperative CSA and results in significantly larger graft area compared to control groups.

TH staining was used to determine cell survival within the graft area. The total number of TH positive cells was calculated for each brain using three series of measurements. Each section in which TH positive cells were detected was included in the evaluation. The total number of TH positive cells in each section was counted and then corrected by the Abercrombie method (See, The Anatomical Record, Vol. 94, pages 239-247, Wistar Institute of Anatomy and Biology, Philadelphia, Pa. 1946) to determine the total number of TH positive cells per striatal VM graft. As seen in FIG. 3, a significant difference in the total number of TH positive cells was observed when the BAF+C5 and Control groups were compared. The difference was shown to be significant using both the Tukey-Kramer and ANOVA (p<0.036) tests. Significantly more TH positive cells were observed in the BAF/C5 cohorts than in the control groups. The results demonstrate that treatment of VM cells with BAF prior to implantation, combined with C5 antibody and postoperative CSA and results in significantly more TH positive (and therefore surviving) cells.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

1. A cell suspension comprising:
cells containing an artificially inflated amount of at least one caspase inhibitor; and
at least one complement inhibitor.

2. The suspension of claim 1 wherein the caspase inhibitor is selected from the group consisting of z-VAD-DCB, z-DEVD-fmk, gene p35, z-VAD.fmk, acetyl-DEVD-CHO, BAF, Ac-YVAD.CMK, Ac-Try-Val-Ala-Asp-aldehyde and crmA.

3. The suspension of claim 1 wherein the caspase inhibitor is bocaspartyl(o-methyl)-flouromethylketone.

4. The suspension of claim 1 wherein the complement inhibitor is selected from the group consisting of anti-C1 antibodies, anti-C2 antibodies, anti-C3 antibodies, anti-C4 antibodies, anti-C6 antibodies, anti-C7 antibodies, anti-C8 antibodies, anti-C9 antibodies, a C5b-9 inhibitor protein, a C-3 inhibitor protein, a chimeric molecule having a C5b-9 inhibitory domain and a C-3 inhibitory domain, a chimeric molecule having a domain having C5b-9 inhibitory activity and a domain having C-3 inhibitory activity, and cobra venum factor.

5. The suspension of claim 1 wherein the complement inhibitor is an anti-C5 antibody.

6. The suspension of claim 1 comprising complement inhibitor in an amount of from about 1 μg/ml to about 1,000 μg/ml of cell suspension.

7. The suspension of claim 1 comprising complement inhibitor in an amount of from about 20 μg/ml to about 500 μg/ml of cell suspension.

8. The suspension of claim 1 comprising complement inhibitor in an amount of from about 50 μg/ml to about 300 μg/ml of cell suspension.

9. The suspension of claim 1 further comprising a pharmaceutically acceptable carrier.

10. A method of treating cellular material to be transplanted comprising
contacting the cellular material to be transplanted with a composition comprising at least one caspase inhibitor, to provide caspase inhibitor-treated transplant material; and
contacting the caspase inhibitor-treated transplant material with a composition containing at least one complement inhibitor.

11. The method of claim 9 wherein the caspase inhibitor is selected from the group consisting of z-VAD-DCB, z-DEVD-fmk, gene p35, z-VAD.fmk, acetyl-DEVD-CHO, BAF, Ac-YVAD.CMK, Ac-Try-Val-Ala-Asp-aldehyde and crmA.

12. The method of claim 9 wherein the caspase inhibitor is bocaspartyl(o-methyl)-flouromethylketone.

13. The method of claim 9 wherein the step of contacting the caspase inhibitor-treated transplant material with a composition containing at least one complement inhibitor comprises contacting the caspase inhibitor-treated transplant material with a composition containing one or more complement inhibitors selected from the group consisting of anti-C1 antibodies, anti-C2 antibodies, anti-C3 antibodies, anti-C4 antibodies, anti-C6 antibodies, anti-C7 antibodies, anti-C8 antibodies, anti-C9 antibodies, a C5b-9 inhibitor protein, a C-3 inhibitor protein, a chimeric molecule having a C5b-9 inhibitory domain and a C-3 inhibitory domain, a chimeric molecule having a domain having C5b-9 inhibitory activity and a domain having C-3 inhibitory activity and cobra venum factor.

14. The method of claim 9 wherein the step of contacting the caspase inhibitor-treated transplant material with a composition containing at least one complement inhibitor comprises contacting the caspase inhibitor-treated transplant material with a composition containing an anti-C5 antibody.

15. The method of claim 9 wherein the step of contacting the caspase inhibitor-treated transplant material with a composition containing at least one complement inhibitor comprises contacting the caspase inhibitor-treated transplant material with a composition containing complement inhibitor in an amount of from about 1 μg/ml to about 1,000 μg/ml of cell suspension.

16. The method of claim 9 wherein the step of contacting the caspase inhibitor-treated transplant material with a composition containing at least one complement inhibitor comprises contacting the caspase inhibitor-treated transplant material with a composition containing complement inhibitor in an amount of from about 20 μg/ml to about 500 μg/ml of cell suspension.

17. The method of claim 9 wherein the step of contacting the caspase inhibitor-treated transplant material with a composition containing at least one complement inhibitor comprises contacting the caspase inhibitor-treated transplant material with a composition containing complement inhibitor in an amount of from about 50 μg/ml to about 300 μg/ml of cell suspension.

18. A method of inhibiting rejection of cellular material to be transplanted in to a recipient comprising:
contacting the cellular material to be transplanted with a composition comprising at least one caspase inhibitor, to provide caspase inhibitor-treated transplant material; and
systemically administering a complement inhibitor to a recipient prior to transplantation of the caspase inhibitor-treated transplant material.

19. The method of claim 18 wherein the step of contacting the cellular material to be transplanted with a composition comprising at least one caspase inhibitor comprises contacting the cellular material to be transplanted with a caspase inhibitor selected from the group consisting of BAF and Ac-YVAD.cmk.

20. The method of claim 18 wherein the step of systemically administering a complement inhibitor comprises administering a composition containing one or more complement inhibitors selected from the group consisting of anti-C1 antibodies, anti-C2 antibodies, anti-C3 antibodies, anti-C4 antibodies, anti-C6 antibodies, anti-C7 antibodies, anti-C8 antibodies, anti-C9 antibodies, a C5b-9 inhibitor protein, a C-3 inhibitor protein, a chimeric molecule having a C5b-9 inhibitory domain and a C-3 inhibitory domain, a chimeric molecule having a domain having C5b-9 inhibitory activity and a domain having C-3 inhibitory activity, a chimeric molecule having a domain having C5b-9 inhibitory activity and a domain having T Cell inhibitory activity, and cobra venum factor.

21. The method of claim 18 further comprising the step of systemically administering one or more immunosuppressive agents selected from the group consisting of cyclosporin A, FK506, rapamycin and a corticosteroid.


SEQUENCE LISTING (3 sequences)

SEQ ID NO: 1 - protein, 4 aa, Artificial Sequence
  Caspase Inhibitor
DEVD

SEQ ID NO: 2 - protein, 4 aa, Artificial Sequence
  Caspase Inhibitor
YVAD

SEQ ID NO: 3 - protein, 4 aa, Artificial Sequence
  Caspase Inhibitor
YVAD